(19) United States
(12) Patent Application Publication
(10) Pub. No.: US 2023/0128598 A1
(43) Pub. Date: Apr. 27, 2023

(54) INHALER

(71) Applicant: RPC Formatec GmbH, Mellrichstadt (DE)

(72) Inventors: Burkard STOCK, Mellrichstadt (DE); Markus THAU, Mellrichstadt (DE)

(21) Appl. No.: 17/971,023

(22) Filed: Oct. 21, 2022

(30) Foreign Application Priority Data
Oct. 21, 2021 (GB) 2115132.9

(51) Int. Cl. A61M 11/02

(52) U.S. Cl. CPC A61M 11/02

(57) ABSTRACT

An actuator for a metered-dose inhaler is provided. The actuator comprises a body for receiving a canister and a metering valve, and a mouthpiece through which aerosolised canister content can be dispensed.

### PRIORITY CLAIM

This application claims priority to GB2115132.9, filed Oct. 21, 2021, which is expressly incorporated by reference herein.

### BACKGROUND

The present disclosure relates generally to inhalers and particularly, although not exclusively, to metered-dose inhalers for delivering an amount of medication to the lungs, in the form of a short burst of aerosolised medicine, that is usually self-administered by a patient via inhalation.

### SUMMARY

According to the present disclosure, a metered-dose inhaler may comprise three major components: a canister (for example produced in aluminium or stainless steel) where the formulation resides; a metering valve, which allows a metered quantity of the formulation to be dispensed with each actuation; and an actuator (or mouthpiece) which allows the user to operate the device and directs the aerosol into the user's lungs. The formulation itself may be made up of a drug, a liquefied gas propellant and, in many cases, stabilizing excipients.

In illustrative embodiments, the user uses the inhaler by pressing down on the top of the canister, with their thumb supporting the lower portion of the actuator. Actuation of the device releases a single metered dose of the formulation which contains the medication either dissolved or suspended in the propellant. Breakup of the volatile propellant into droplets, followed by rapid evaporation of these droplets, results in the generation of an aerosol comprising micrometer-sized medication particles that are then inhaled.

In illustrative embodiments, an actuator for a metered-dose inhaler comprises a body for receiving a canister and a metering valve, and a mouthpiece through which aerosolised canister content can be dispensed, the body comprises a support block which comprises a metering valve stem receptacle and a sump which leads, via a nozzle bore, to a funnel-shape nozzle opening leading to the mouthpiece, the sump has a generally triangular cross section, with a proximal end of an inclined face extending immediately adjacent the receptacle and from which the nozzle bore extends generally centrally and towards, but not at, a distal end of the inclined face.

The axis of the nozzle bore may be generally perpendicular to the inclined face.

In illustrative embodiments, the important dimensions are depth and diameter of a pre-chamber.

In illustrative embodiments, the sump could be thought of as generally an oblique cone with a non-circular section and a generally rectangular frontal and/or posterior elevation.

In illustrative embodiments, the actuator may provide a sump volume of approximately 12.6 mm³.

In illustrative embodiments, the actuator may provide a sump volume of approximately 20.2 mm³.

In illustrative embodiments, the actuator may provide a sump volume of approximately 12.7 mm³.

In illustrative embodiments, the actuator may provide a sump volume of approximately 15.3 mm³.

In illustrative embodiments, the actuator or parts thereof may be formed substantially as shown in one or more the drawings.

In illustrative embodiments, the actuator may have a geometry substantially as shown in FIG. 3.

In illustrative embodiments, the actuator may have a geometry substantially as shown in FIG. 6.

In illustrative embodiments, the actuator may have a geometry substantially as shown in FIG. 7.

In illustrative embodiments, the actuator may have a geometry substantially as shown in FIG. 8.

In illustrative embodiments, the actuator may have a geometry substantially as shown in FIG. 9.

In illustrative embodiments, a dimension measured from the top of the support block to the bottom of the sump may be generally between 1.80 mm and 2.20 mm, for example 1.8 mm±0.05 mm or 2.20±0.05 mm or 1.95 mm±0.05 mm.

In illustrative embodiments, a dimension measured as the diameter or width at the mouth of the sump may be between 8.25 mm and 9.70 mm, for example 9.70 mm±0.20 mm or 8.25 mm±0.20 mm.

In illustrative embodiments, combinations of the above dimensions are possible to and may be used to provide different sump volumes.

In illustrative embodiments, the sump may have a generally wedge-shape section.

In illustrative embodiments, the sump may be open at one end and closed at the other end by a curved apex.

In illustrative embodiments, the nozzle bore may extend laterally centrally from the inclined face.

In illustrative embodiments, the inclined face may be generally rectangular.

In illustrative embodiments, a metered-dose inhaler comprises an actuator as described and/or defined herein.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a perspective view of an inhaler in accordance with the present disclosure;

FIG. 2 is a perspective view of an inhaler in accordance with the present disclosure with portions broken away;

FIG. 3 is a side elevation sectional view of an inhaler in accordance with the present disclosure;

FIG. 4 is front elevation sectional view of an inhaler in accordance with the present disclosure;

FIG. 5A is a front elevation view of an inhaler in accordance with the present disclosure;

FIG. 5B is a sectional view taken along line A-A of FIG. 5A;

FIG. 5C is an enlarged view of a portion of FIG. 5B;

FIG. 5D is a sectional view taken along line B-B of FIG. 5A;

FIG. 5E is a an enlarged view of a portion of FIG. 5D;

FIG. 5F is a bottom plan view of the inhaler of FIG. 5A;

FIG. 5G is an enlarged view of a portion of FIG. 5B;

FIG. 5H is an enlarged view of a portion of FIG. 5B;

FIG. 5I is an enlarged view of a portion of FIG. 5C;

FIG. 5J is a perspective view of the inhaler of FIG. 5A;

FIG. 6 is an enlarged view of a portion of an inhaler with sump volume of about 12.6 mm³ and some dimensions shown;

FIG. 7 is an enlarged view of a portion of an inhaler with sump volume of about 20.2 mm³ and some dimensions shown;

FIG. 8 is an enlarged view of a portion of an inhaler with sump volume of about 12.7 mm³ and some dimensions shown;

FIG. 9A is a front elevation view of an inhaler in accordance with the present disclosure showing some dimensions;

FIG. 9B is a sectional view taken along line A-A of FIG. 9A showing some dimensions;

FIG. 9C is an enlarged view of a portion of FIG. 9B showing some dimensions;

FIG. 9D is a sectional view taken along line B-B of FIG. 9A; showing some dimensions;

FIG. 9E is a bottom plan view of the inhaler of FIG. 9A showing at least one dimension;

FIG. 9F is an enlarged view of a portion of FIG. 9B showing some dimensions;

FIG. 9G is an enlarged view of a portion of FIG. 9C showing some dimensions; and

FIG. 9H is a perspective view of the inhaler of FIG. 9A.

### DETAILED DESCRIPTION

The present disclosure will now be more particularly described, by way of example, with reference to the accompanying drawings.

Example embodiments are shown and described in sufficient detail to enable those of ordinary skill in the art to embody and implement the systems and processes herein described. It is important to understand that embodiments can be provided in many alternate forms and should not be construed as limited to the examples set forth herein. Drawings are drawn to scale.

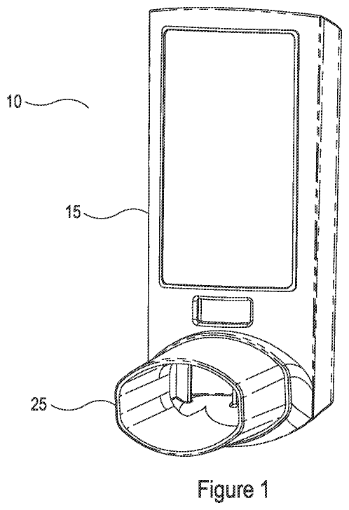

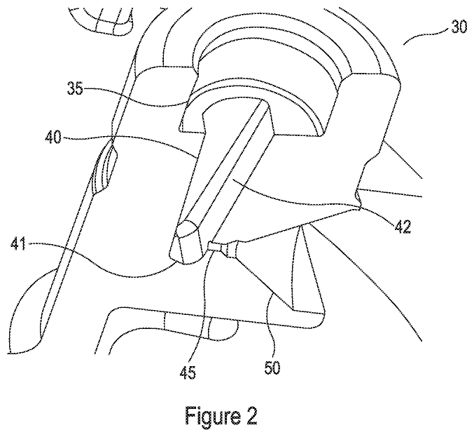

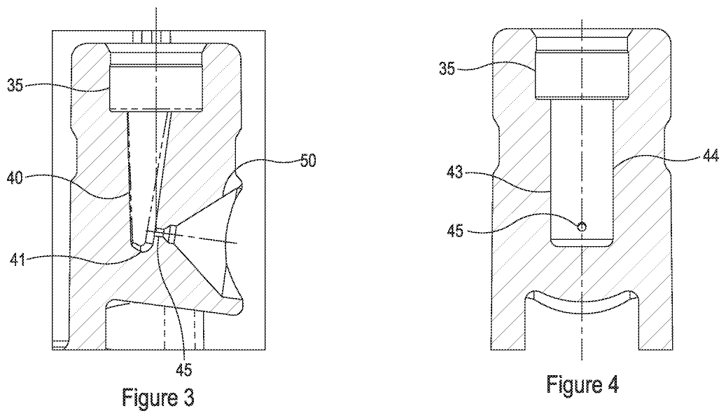

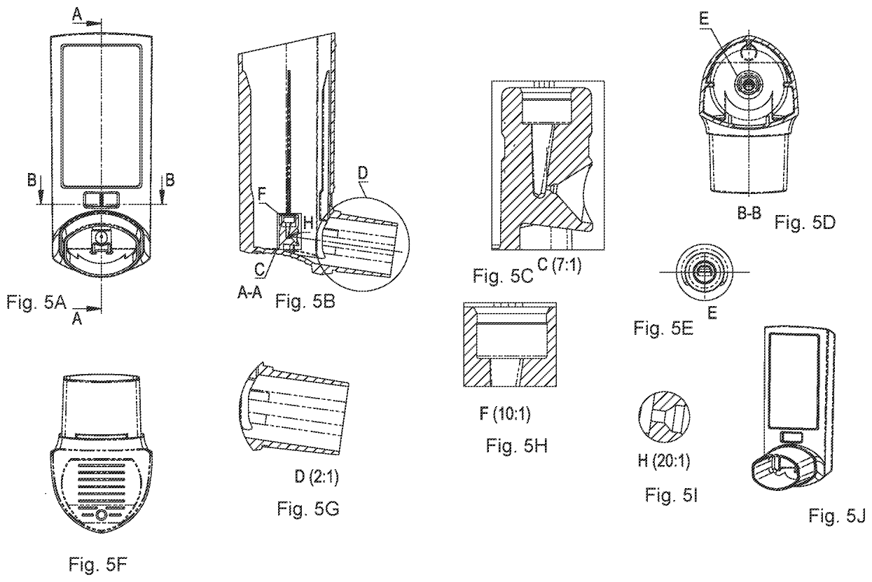

FIGS. 1 to 4 illustrate a metered-dose inhaler formed in accordance with an aspect of the present disclosure.

The inhaler (10) comprises a body (15) for receiving a canister (not shown) and a metering valve (20), and a mouthpiece (25) through which aerosolised canister content can be dispensed.

The body comprises a support block (30) which comprises a metering valve stem receptacle (35) and a sump (40) which leads, via a nozzle bore (45), to a funnel-shape nozzle (50) opening leading to the mouthpiece. In some embodiments the stem receptacle is considered to form part of the sump for the purposes of calculating the sump volume.

The sump (40) has a generally wedge-shape, triangular (e.g. isosceles or scalene) cross-section with a curved apex (41) and open at its other end, and with a proximal end of an inclined face (42) extending immediately adjacent the receptacle and from which the nozzle bore extends generally centrally and towards, but not at, a distal end of the inclined face. The axis (X) of the nozzle bore (45) is generally perpendicular to the inclined face (42).

As shown best in FIG. 4, when viewed “face on” the sump (40) is generally rectangular in section and has generally parallel sides (43, 44). The nozzle bore (45) is positioned generally centrally with respect to the sump.

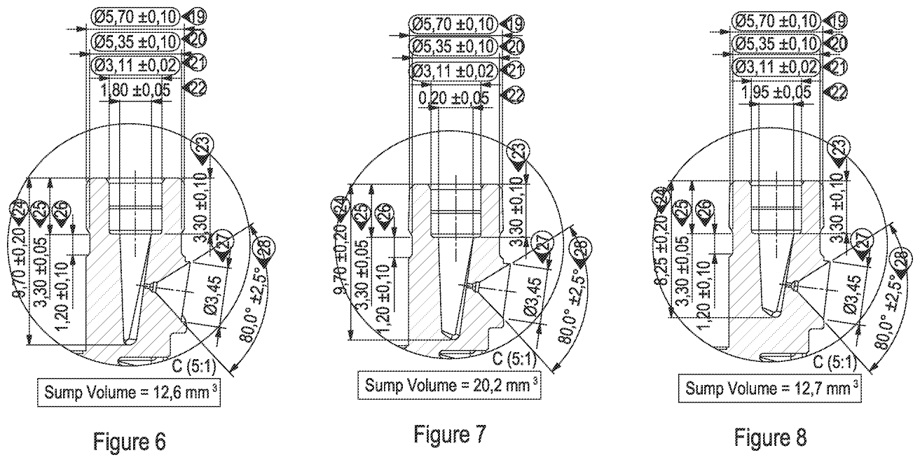

FIGS. 6, 7 and 8 show support blocks formed in accordance with further embodiments of the present disclosure.

The dimensions marked in red may vary.

The dimension for reference numeral 22 may be between 1.80 mm and 2.20 mm. This dimension may be measured from the “top” of the support block to the “bottom” of the sump.

The dimension for reference numeral 24 may be between 8.25 mm and 9.70 mm. This dimension may be measured as the diameter/width at the mouth of the sump.

The support block of FIG. 6 provides a sump volume of approximately 12.6 mm³.

The support block of FIG. 7 provides a sump volume of approximately 20.2 mm³.

The support block of FIG. 6 provides a sump volume of approximately 12.7 mm³.

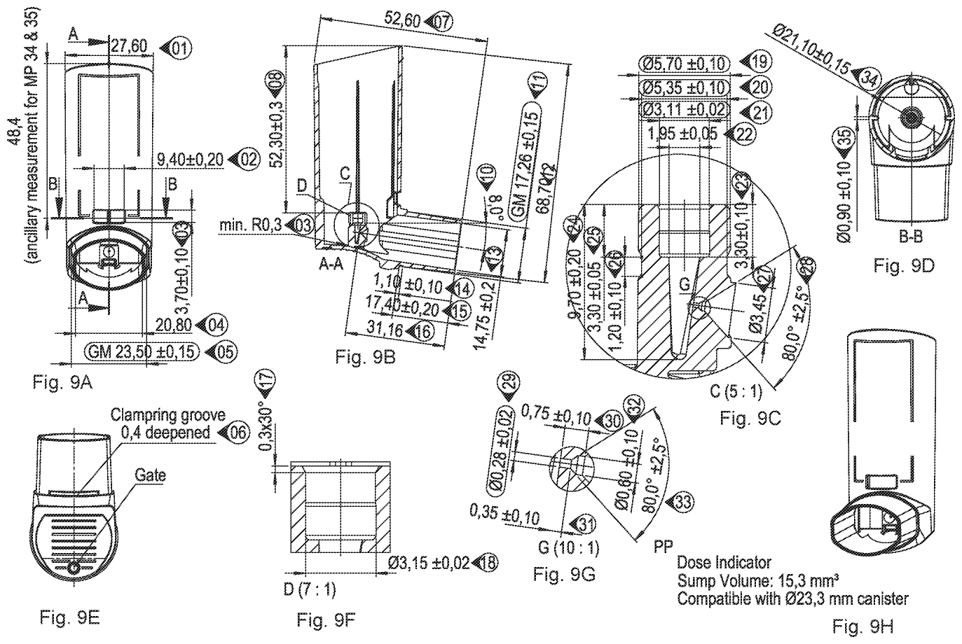

FIG. 9 shows an inhaler body formed in accordance with a further embodiment.

The support block of FIG. 9 provides a sump volume of approximately 15.3 mm³.

An actuator for a metered-dose inhaler is provided. The actuator comprises a body for receiving a canister and a metering valve, and a mouthpiece through which aerosolised canister content can be dispensed. The body comprises a support block which comprises a metering valve stem receptacle and a sump which leads, via a nozzle bore, to a funnel-shape nozzle opening leading to the mouthpiece. The sump has a generally triangular cross section, with a proximal end of an inclined face extending immediately adjacent the receptacle and from which the nozzle bore extends generally centrally and towards, but not at, a distal end of the inclined face.

Although illustrative embodiments of the present disclosure have been disclosed in detail herein, with reference to the accompanying drawings, it is understood that the present disclosure is not limited to the precise embodiments shown and that various changes and modifications can be effected therein by one skilled in the art without departing from the scope of the present disclosure as defined by the appended claims and their equivalents.

1. An actuator for a metered-dose inhaler, the actuator comprising a body for receiving a canister and a metering valve, and a mouthpiece through which aerosolised canister content can be dispensed, the body comprises a support block which comprises a metering valve stem receptacle and a sump which leads, via a nozzle bore, to a funnel-shape nozzle opening leading to the mouthpiece, the sump has a generally triangular cross section, with a proximal end of an inclined face extending immediately adjacent the receptacle and from which the nozzle bore extends generally centrally and towards, but not at, a distal end of the inclined face.

2. The actuator of claim 1, in which the axis of the nozzle bore is generally perpendicular to the inclined face.

3. The actuator of claim 1, providing a sump volume of approximately 12.6 mm³.

4. The actuator of claim 1, providing a sump volume of approximately 20.2 mm³.

5. The actuator of claim 1, providing a sump volume of approximately 12.7 mm³.

6. The actuator of claim 1, providing a sump volume of approximately 15.3 mm³.

7. The actuator of claim 1, in which a dimension measured from the top of the support block to the bottom of the sump is between 1.80 mm and 2.20 mm.

8. The actuator of claim 7, in which the dimension is 1.8 mm±0.05 mm.

9. The actuator of claim 7, in which the dimension is 2.20 mm±0.05 mm.

10. The actuator of claim 7, in which the dimension is 1.95 mm±0.05 mm.

11. The actuator of claim 1, in which a dimension measured as the diameter or width at the mouth of the sump is between 8.25 mm and 9.70 mm.

12. The actuator of claim 11, in which the dimension is 9.70 mm±0.20 mm.

13. The actuator of claim 11, in which the dimension is 8.25 mm±0.20 mm.

14. The actuator of claim 1, in which the sump has a generally wedge-shape section.

15. The actuator of claim 1, in which the sump is open at one end and closed at the other end by a curved apex.

16. The actuator of claim 15, in which the nozzle bore extends laterally centrally from the inclined face.

17. The actuator of claim 1, in which the inclined face is generally rectangular.

18. A support block for the inhaler of claim 1.

19. A metered-dose inhaler comprising the actuator of claim 1.
